(19) United States
(12) Patent Application Publication
(10) Pub. No.: US 2013/0144335 A1
(43) Pub. Date: Jun. 6, 2013

(54) SUTURE ANCHOR

(71) Applicant: Michael John Sandow, Adelaide (AU)

(72) Inventor: Michael John Sandow, Adelaide (AU)

(21) Appl. No.: 13/578,798

(22) PCT Filed: Feb. 9, 2011
(86) PCT No.: PCT/AU11/00131

§ 371 Date: Dec. 6, 2012

(30) Foreign Application Priority Data
Feb. 9, 2010 (AU) 2010900613

(51) Int. Cl. A61B 17/04

(52) U.S. Cl. CPC A61B 17/0401
USPC 606/232

(57) ABSTRACT

When ligaments or tendons detach from associated bone, or become damaged by way of being torn, surgical intervention can be used to assist repair and reattachment (restoration). An anchor in the cortical portion of the bone is provided which is used to secure a suture with respect to the bone wherein the suture is attached at one end to a tendon or other portion of the body and the free end is secured to the anchor without the use of a knot. An arthroscopic transosseous knotless suture anchoring arrangement for holding a suture includes, a hollow body for at least partial insertion into a cortical portion of a hone and a holding dement having an elongate body sized for fitment with and within the hollow of the body for holding a suture located in the hollow body with respect to the cortical portion of the bone.

### CROSS-REFERENCE TO RELATED U.S. APPLICATIONS

Not/applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

This invention relates generally to an apparatus used in the securement of the position of a suture.

2. Description of Related Art Including Information Disclosed. Under 37 CER 1.97 and 37 CER 1.98.

When ligaments or tendons detach either fully or partially from associated bone, or become damaged by way of being torn, surgical intervention can be used to assist repair and reattachment (restoration). While both open and arthroscopic surgical techniques can he used, the surgeon's clinical assessment of the nature of problem, the restoration technique options, the associated surgical elements (sutures, anchors, tools required), and accessibility to the area and associated areas of the restoration, will determine the actual surgical approach.

Arthroscopic surgical techniques are at this time increasingly preferred for multiple reasons, such as increased access and potential ease of use of surgical elements during surgery, less disturbance of the patient's body organs and tissues, and typically reduced recovery periods (of particular importance to sports persons) during which there is less discomfort for the patient.

Unfortunately, intra-corporeal suturing is difficult and hence time-consuming, and only basic knots and stitch patterns can be used. Furthermore, the tension applied to the suture and hence to the ligaments and tendons attached thereto-can be difficult to maintain during the knot tying procedure, as also can achieving acceptable tightness of the knots. Also, it is usually not. possible to adjust either the tension of the suture or the knot once formed. Yet further, the knots and knot bundles that secure the suture are by necessity proud of the restoration, region (thus post operatively the patient may feel the knot bundle or bundles) which can create a potential source of irritation and increased scarring during the healing process. Open surgical techniques allow for the full spectrum of suturing, knotting, and knot placement, although palpation of the knot or knot bundles is still, possible in some cases depending on the location of the surgery.

A wide range of anchors are used for glenohumeral instability, superior labrum anteroposterior injury, and rotator cuff repairs, and those anchors are generally used for fixing the ends of sutures to the bony structure of the body. However, there are a wide range of problems associated with these anchors and the way they achieve that attachment, to the bone and to the suture/s. Anchors including a screw portion are known for providing a mechanical bond to bone. The difference in density in the cortical bone (the relatively dense and hard outer layer of bone) and the cancellous bone (the relatively less dense, soft and somewhat density health of the bone, to the diameter of any pilot hole made by the surgeon, and the ability to grow about and sometimes over the anchor. Furthermore, this type of anchor can typically only he inserted in one action and does not provide for adjustability of depth or angle.

Another approach to hone anchor design uses a split hollow shaft inserted into a hole created in the cortical bone. The anchor carries a tapered plug extending through the full length of the hollow shaft of the anchor with the broadest end of the inner end (with respect to the hone) of the shaft located such that when the plug is pulled towards the outer end of the shaft the tapered portion of the plug causes the hollow shaft body to flare radially outward effectively locking the device into the cortical bone structure. This type of anchor also needs to be inserted in one action and does not provide for adjustability of depth or angle.

Anchors of the type described in the previous paragraph can he used in suitable bones, and if used in conjunction with sutures, will fixedly incorporate typically two or more lengths of suture material which are then available for attachment to the relevant tissue and securely knotted as required. An alternative is to provide an eyelet on the outer end of the anchor through which suture material can be threaded and/or tied to. A problem with such anchor types is that the eyelet can break or unduly abrade the suture material in addition to the time and skill involved hi placing the suture material into the soft tissue, looping through the anchor eyelet, and tying knots wherein all these procedures are done during arthroscopic surgery. This type of anchor also is preferably inserted in one action and does not provide for adjustability of depth or angle.

Other methods of securing soft tissue to bone such as staples, tacks and the like are known to fail when tension is applied or work themselves loose and can cause inflammation, irritation, or not be suitable for some locations in the body, and furthermore, be difficult to post operatively locate when they have dislodged.

### BRIEF SUMMARY OF THE INVENTION

In a broad aspect of the invention in arthroscopic transosseous knotless suture anchoring arrangement for holding a suture with respect to a cortical portion of a bone, includes, a hollow body for at least partial insertion into a cortical portion of a bone; and a holding element having an elongate body sized for fitment with and within the hollow of the body for holdings portion of a suture located in the hollow body with respect to the cortical portion of the bone.

In another aspect of the invention a device for capturing and holding a suture under tension with respect to a cortical bone portion includes, a hollow elongate body having an inner surface forming the hollow, at least a portion of the body for placement in a cavity located in the cortical bone portion, the hollow body having a rim radially extending from one end of the body, the rim adapted to receive a force that forces at least a portion of the rim to abut the cortical bone adjacent to the cavity in the cortical bone; and a holding element sized for fitment with and within the hollow of the body: wherein a portion of the suture is located within the hollow body and while, the free end of the suture is under tension caused by a force on the suture applied in a direction away from the outer surface of the cortical bone, and another force in substantially the opposite direction is applied to the rim of the body, the holding element is interference fitted into the hollow body so as to capture a portion of the suture under tension which is located within the hollow body while the run of the body abuts the cortical bone portion and also holds the captured portion of the suture with respect to the cortical bone portion.

Throughout this specification and the claims that follow unless the context requires otherwise, the words ‘comprise’ and ‘include’ and variations such as ‘comprising’ and ‘including’ will be understood to imply the inclusion of a stated integer or group of integers but not the exclusion of any other integer or group of integers.

The reference to any prior art in this specification is not, and should not be taken as, an acknowledgment or any form of suggestion that such prior art forms part of the common general knowledge.

A detailed description of one or more preferred embodiments of the invention is provided below along with accompanying Figures that illustrate by way of example the principles of the invention. While the invention is described in connection with such embodiments, it should be understood that the invention is not limited to any embodiment. On the contrary, the scope of the invention is limited only by the appended claims and the invention encompasses numerous alternatives, modifications and equivalents. For the purpose of example, numerous specific details are set forth in the following description in order to provide a thorough understanding of the present invention. The present invention may be practiced according to the claims without some or all of these specific details. For the purpose of clarity, technical material that is known in the technical fields related to the invention has not been described in detail so that the present invention is not unnecessarily obscured.

### BRIEF DESCRIPTION OF THE DRAWINGS

FIG. 1 depicts a breakaway anterior view of a muscular-skeletal view of a rotator cuff of a human;

FIG. 2 depicts a breakaway view of a torn rotator cuff;

FIG. 3 depicts a suture attachment to the rotator cuff through a lateral skin aperture;

FIG. 4 depicts inspection and possible preparation of the top of the numerous through the lateral skin aperture including the making of a hole or trench in a humerus bone;

FIG. 5 depicts a suture transfer guide instrument being inserted through one of the arthroscopic skin apertures and into a hole in the upper humerus, and being positioned in preparation for creating the lateral humeral hole;

FIG. 6 depicts the trochar and suture retrieval sleeve being positioned and about to be inserted into the guiding sleeve on the outer arm of the suture transfer guide;

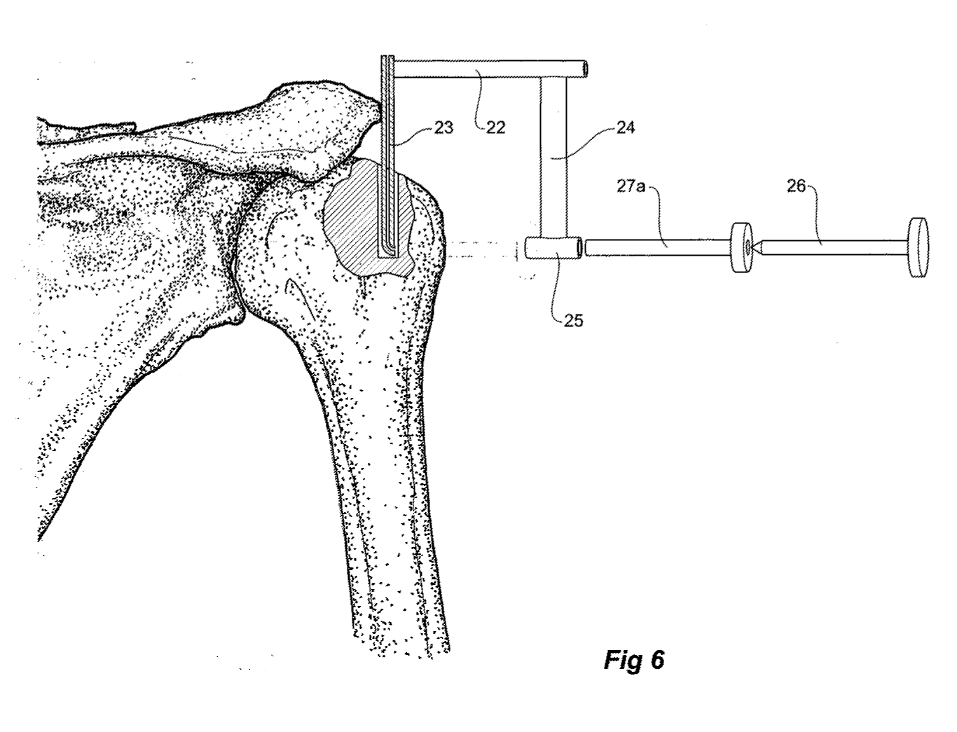

FIG. 7 depicts the trochar inserted into the suture retrieval sleeve and about to be inserted into the guiding sleeve on the outer arm of the suture transfer guide;

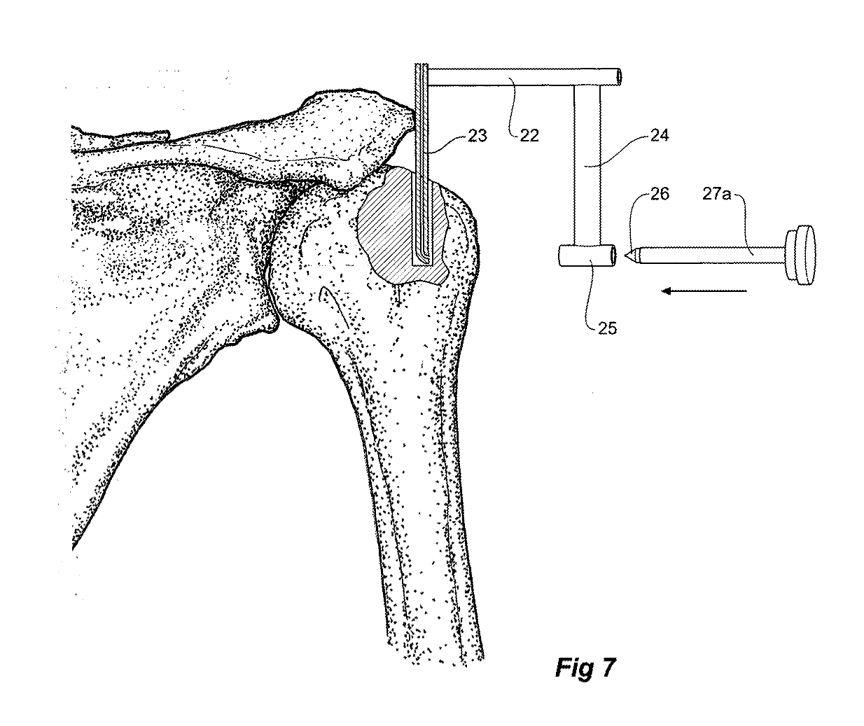

FIG. 8 depicts the suture retrieval sleeve with its trochar being advanced, though the lower skin aperture, through a hole in the lateral aspect of the humerus and into the humeral bone medulla to a point adjacent to the intra-osseous portion, of the suture transfer guide;

FIG. 9 depicts the withdrawal of the trochar, leaving the suture retrieval sleeve in place;

FIG. 10 depicts the suture transfer guide in place with the suture retrieval sleeve in place adjacent to the foramen at the distal portion of the intra-osseous portion of the guide;

FIG. 11 depicts the threading of a guide thread through the central hole in the intra-osseous portion of the suture transfer guide and then out through the suture retrieval sleeve so it passes through both holes in the humerus;

FIG. 12 depicts the withdrawal of the suture retrieval sleeve, but leaving the guide thread in place;

FIG. 13 depicts the removal of the intra-osseous arm of the guide suture transfer from the humerus, taking care that the guide thread remains in place;

FIG. 14 depicts the disengagement of the suture transfer guide from the guide thread;

FIG. 15 depicts both the suture attached to the rotator cuff and the guide thread threaded through the bone;

FIG. 16 depicts the suture after being pulled through the hole in the bone still attached to the rotator cuff;

FIG. 17 depicts the rotator cuff being pulled by the suture through one of the holes in the bone;

FIG. 18 depicts the threading of a hollow body over the suture;

FIG. 19 depicts the embedding of the hollow body into the bone;

FIG. 20 depicts the placement of a holding element through one of the two lateral skin openings towards the hollow body;

FIG. 21 depicts the placement of the holding element into the hollow body;

FIG. 22 depicts fitment of the holding element and the repair suture/s within the hollow body, and with the sutures divided at the outer surface of the holding body;

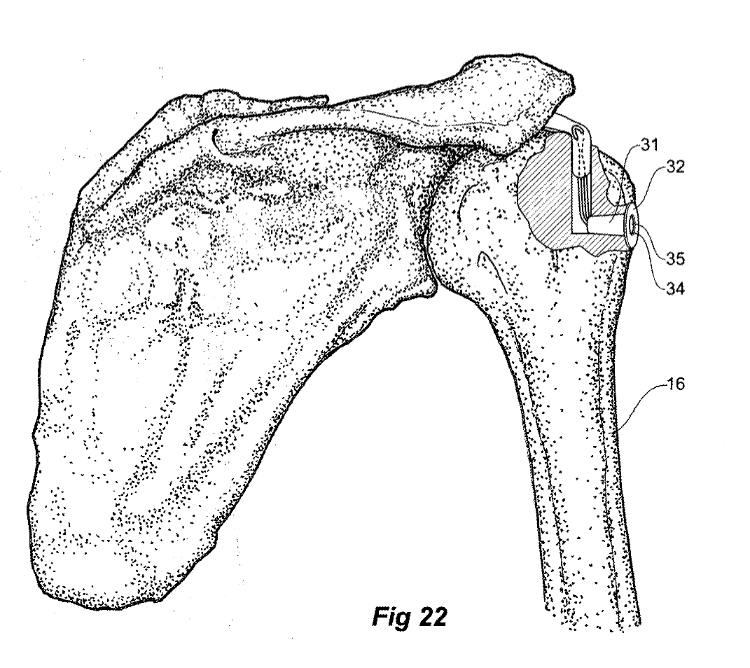

FIG. 23 depicts a guide to assist, the feeding of the holding element into the hollow body using a plug and the guide also impacting the hollow body into the bone;

FIG. 24 depicts pictorial representations of a holding element and a hollow body;

FIG. 25 depicts a pictorial representation of the positioning, of the holding element while two sutures are threaded through the hollow body;

FIG. 26 depicts a pictorial representation of the insertion of the holding element into the hollow body capturing the sutures; and

FIG. 27 depicts a pictorial representation of the fitted position of the holding element within the hollow body, and capturing the repair sutures.

### DETAILED DESCRIPTION OF THE INVENTION

The devices described herein are useable to secure sutures in bone. There are many applications for the devices disclosed which are within the skill of a surgeon to determine. However, there is only one example of the use of the devices disclosed herein, which relates to the repair and remediation of damage to the rotator cuff using arthroscopic surgical procedures.

The rotator cuff comprises muscles and their tendons including the subscapularis, supraspinatus, infraspinatus and teres minor, which surround the shoulder and connect between the humerus and the scapula. They will attach along the head of the humerus and their primary role is to help stabilise the shoulder during active movement, which is generally under the action of the deltoid muscle.

All the muscles must work together, contracting and relaxing in synchronisation in order to provide a stable and mobile shoulder through the required range of motion.

The stated procedures used by way of example herein include tensioning and positioning of the pathologically detached rotator cuff back on to prepared bone.

Following an inspection and determination of the extent of damage or disease of the rotator cuff and surrounding tissue and bone, a clean up of the rotator cuff precedes a clean up of the bone in preparation for surgical procedures to assist the apposition of connective tissue to the prepared bone.

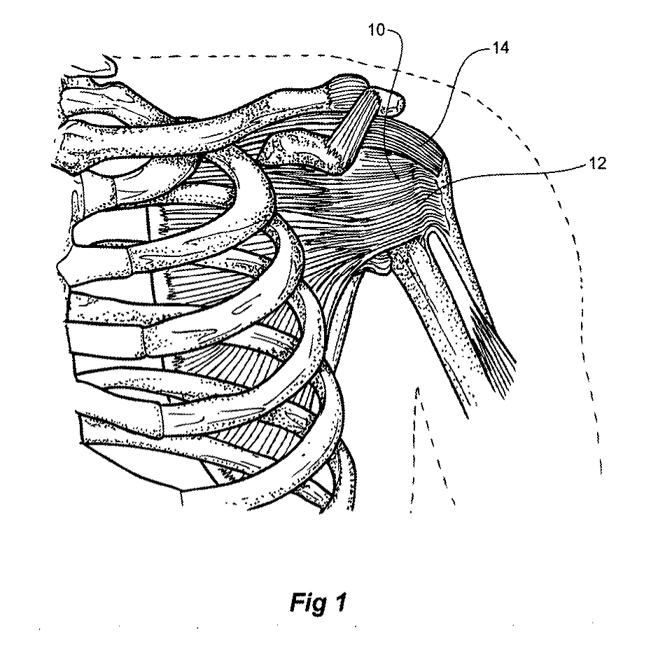

FIG. 1 depicts a breakaway anterior view of a muscular-skeletal view of a rotator cuff of a human.

The rotator cuff group of muscles is depicted at 10 with the subscapularis 12 and supraspinatus 14 as shown in FIG. 1.

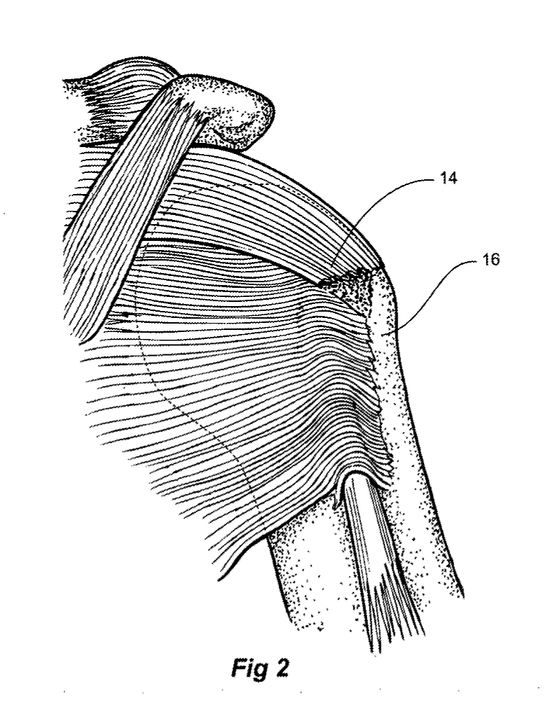

FIG. 2 depicts a breakaway view of a torn rotator cuff wherein the supraspinatus 14 is shown partially separated from the humerus 16.

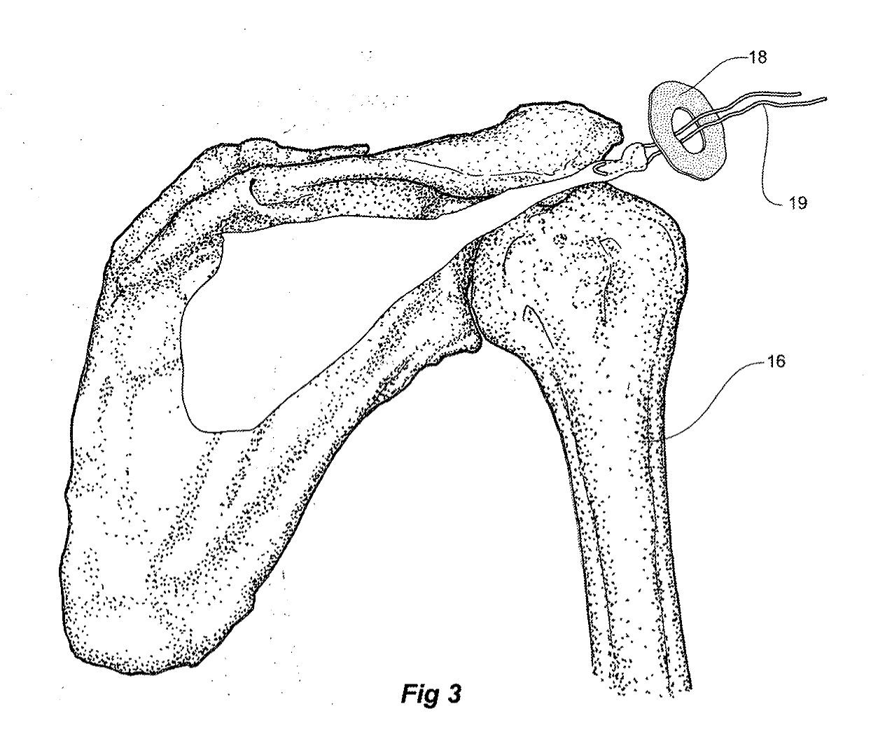

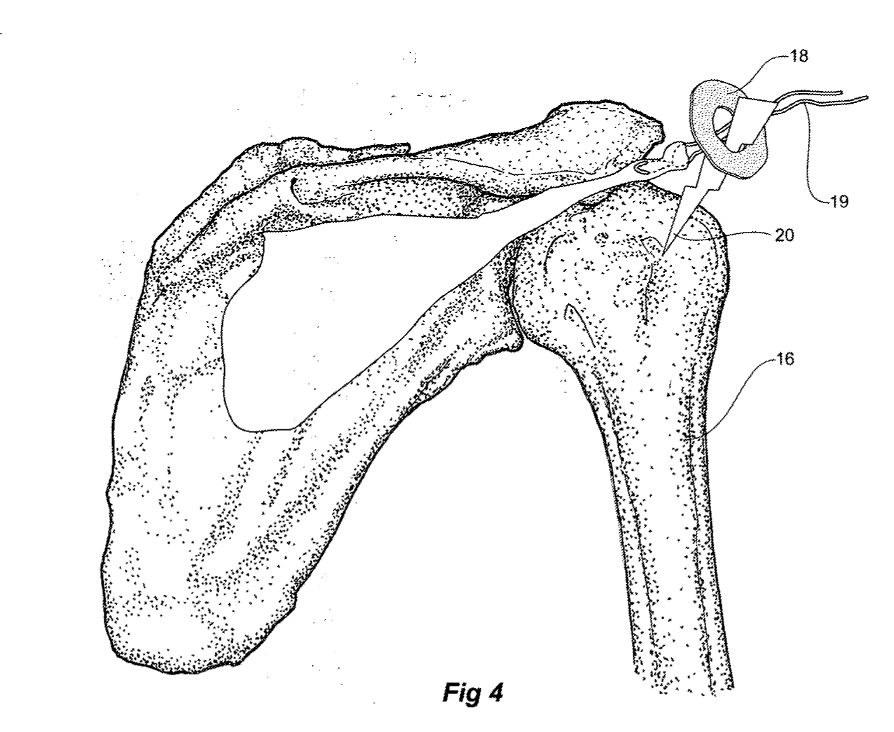

As stated, arthroscopic surgery is to be used and in preparation for the surgery, openings in the skin of the patient are made through which the various procedures are conducted. In FIG. 3 a pictorial representation of the aperture is shown as 18. There is typically more than one aperture created for the surgery, but only one is depicted pictorially in FIG. 3. Both an high skin aperture 18 (FIGS. 3 and 4) and lower on the upper arm skin an aperture 29 is shown in Figure S and these apertures remain the only access to the deeper tissues and humerus during the procedure. They are not included in other Figures for diagrammatic simplicity, but it is inherent in the procedure described, that surgical access is only via these specific apertures.

FIG. 3 depicts a suture attachment to the torn tendon of the infraspinatus muscle of the rotator cuff muscles showing in particular the suture 19 drawn to the exterior of the patient through the aperture 18 to be external to the patient's body. Later in the procedure a suture or guide thread 28 will also be drawn out through the high skin aperture 18, which will allow the guide thread to be attached to the repair suture 19 as detailed below.

FIG. 4 depicts inspection and possible preparation of the top of the numerous through the lateral skin aperture 18 and includes the making preparations for the making of a hole or trench in the humerus bone 16.

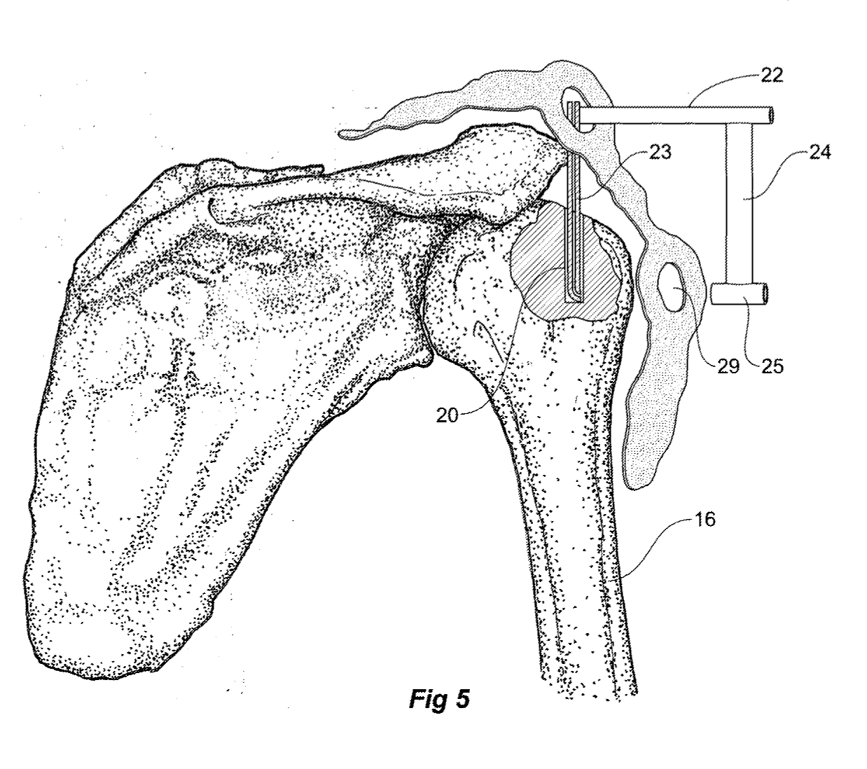

FIG. 5 depicts, the preliminary step of making a hole or trench 20 in the upper region of the humerus, suitable to receive the suture transfer guide post 23. The hole 20 to be made is the first of two holes to be made in the humerus bone and the second hole needs to be made so as to meet up with the first hole inside the bone and thus to provide a passage through the bone.

FIG. 5 also pictorially illustrates how it is determined where both the first and second holes in the humerus bone should he made using a suture transfer guide instrument. The suture transfer guide instrument has a guide post 23 inserted through aperture 18 in the skin of the patient and then under arthroscopic visual control, inserted, into the trench/first hole 20.

The suture transfer guide instrument has an second guide arm 24 which is located parallel to the guide post 23 by the first guide arm 22, at a sufficient distance between the guide post 23 and the second guide arm 24 to allow for the thickness of soft tissue, including the deltoid muscle, subcutaneous fat and skin over the bone. At the bottom of the second guide arm 24 is a hollow cylindrical shaped attachment 25 at a right angle to the second guide arm 24 and of sufficient bore size and shape to allow passage of the longitudinal trochar 26 and suture retrieval sleeve 27a yet to be described.

The guide post 23 has a bore its full length that is of sufficient size to allow easy passage of a guide thread 28 or suture that has stiffness sufficient to allow it to feed without coiling or kinking (by way of example, stiff plastic coated braided wire or thread). The hole at the bottom of the guide post 23 is at a right angle to the long axis of the guide post 23 and directed laterally towards the second guide arm 24 and specifically in line with the centre of the guiding sleeve 25.

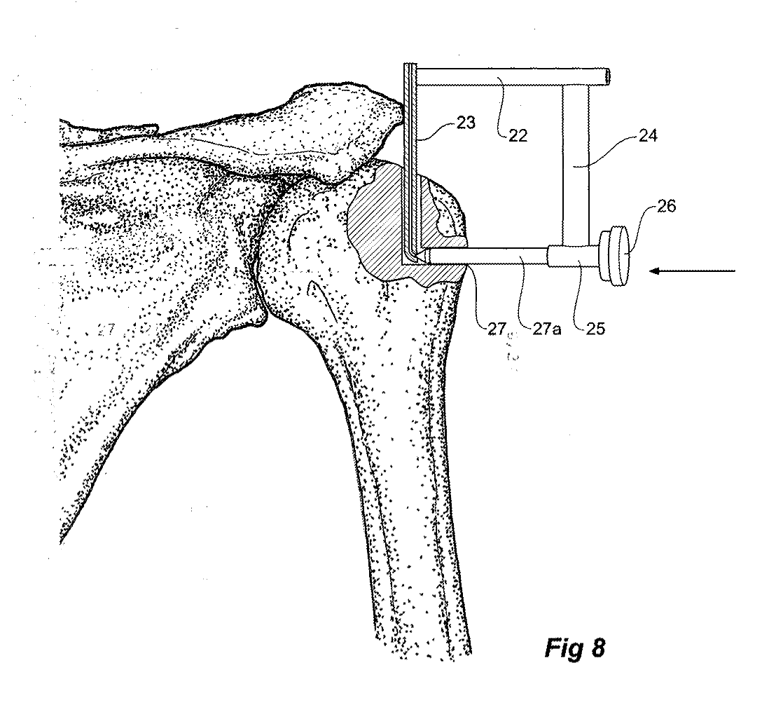

Placement of the guide post 23 within the intended hole 20 and positioning of the second guide arm 24 at or near the top of the humerus bone near where the second hole 27 (FIG. 8) in the humerus is to be created are a judgment call by the surgeon.

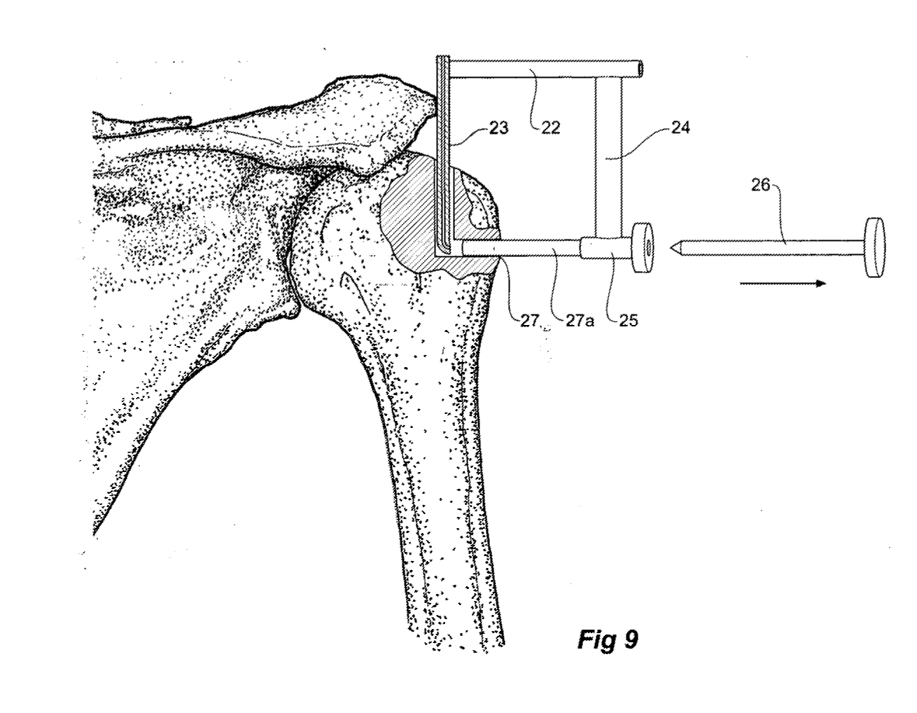

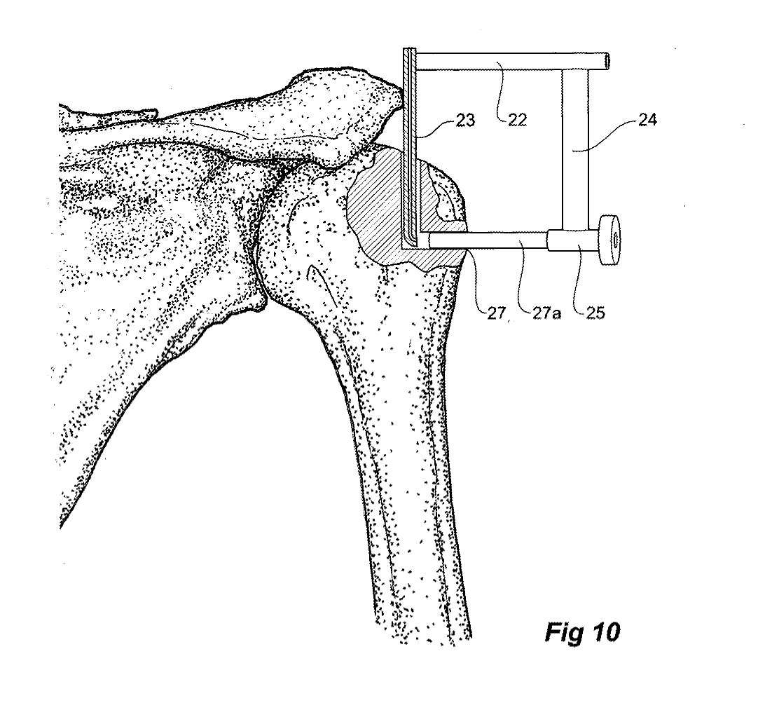

The process of making the second hole 27 (FIG. 8) is achieved, by placing a trochar 26 and suture retrieval sleeve 27a and guiding sleeve 25 and making the hole 27 in the numerous through the cortical hone as pictorially depicted in FIG. 8. Once the second hole is made, the trochar 26 and suture retrieval sleeve 27a is advanced within the humerus until in contact with the first post 23. The trochar 26 is then removed as is depicted in FIG. 9 and the suture retrieval sleeve 27a left in place (FIG. 10).

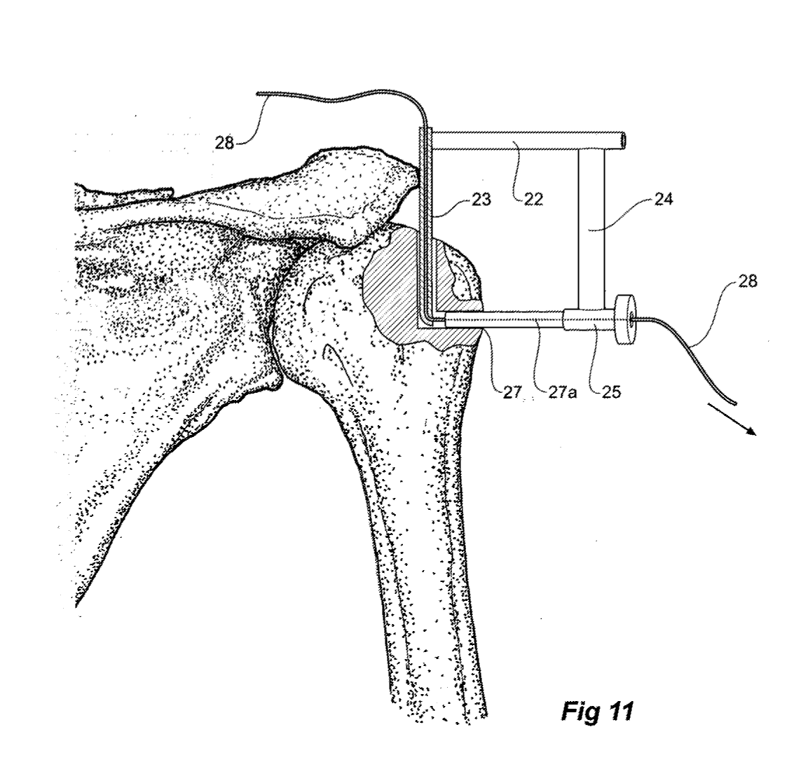

As depicted in FIG. 11, stiff suture or fine guide thread 28 is inserted into the upper externally exposed hole of the guide post 23 and advanced into the bore. As the hole at the bottom of the guide post 23 is substantially in line with the centre of the suture retrieval sleeve 27a, the suture or guide thread 28 advances through the suture retrieval sleeve 27a to exit at its lateral end. The suture or guide thread 28 is then advanced a sufficient distance to ensure it can be secured at both ends external of the body.

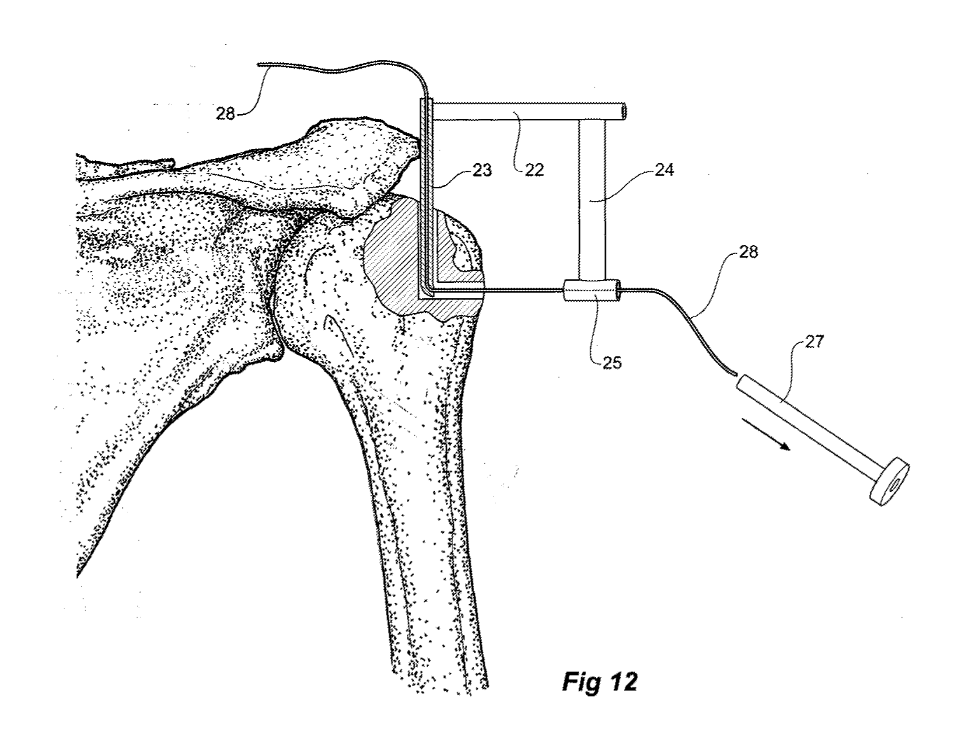

As depicted in FIG. 12 the suture retrieval sleeve 27a is then withdrawn laterally until it clears the guiding sleeve 25 and is slid off the suture or guide thread 28, ensuring the suture or guide thread 28 remains present at both the upper 18 and lower 29 skin apertures (not shown).

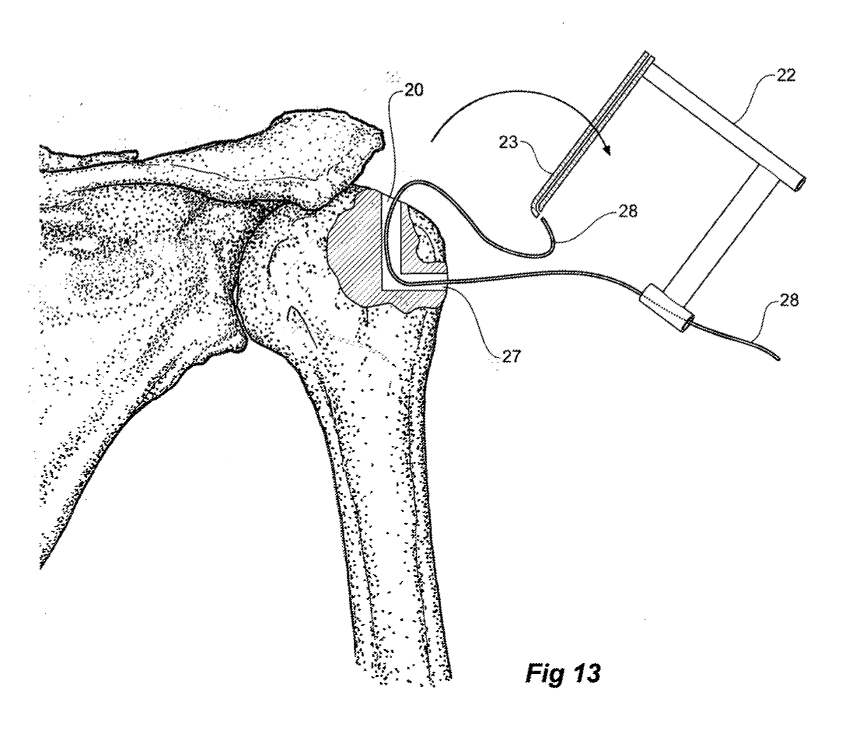

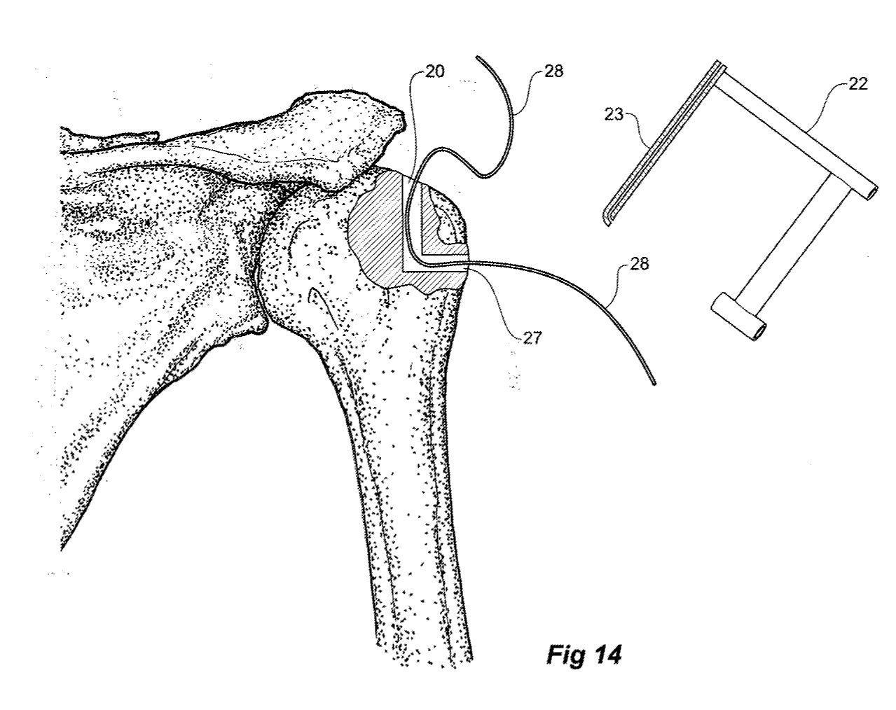

The guide post 23, first guide arm 22 and second guide arm 24 are together then disengaged from the trench 20 in the humerus 16 and withdrawn from the upper skin aperture 18. The guide post 23 is then slid off the suture or guide thread 28 (FIG. 13) initially and then the guiding sleeve 25 is slid off the suture or guide thread (FIG. 14).

FIG. 14 depicts a suture or guide thread 28 passing through the passage created by two holes in the humerous via the higher skin aperture 18 and lower skin aperture 29.

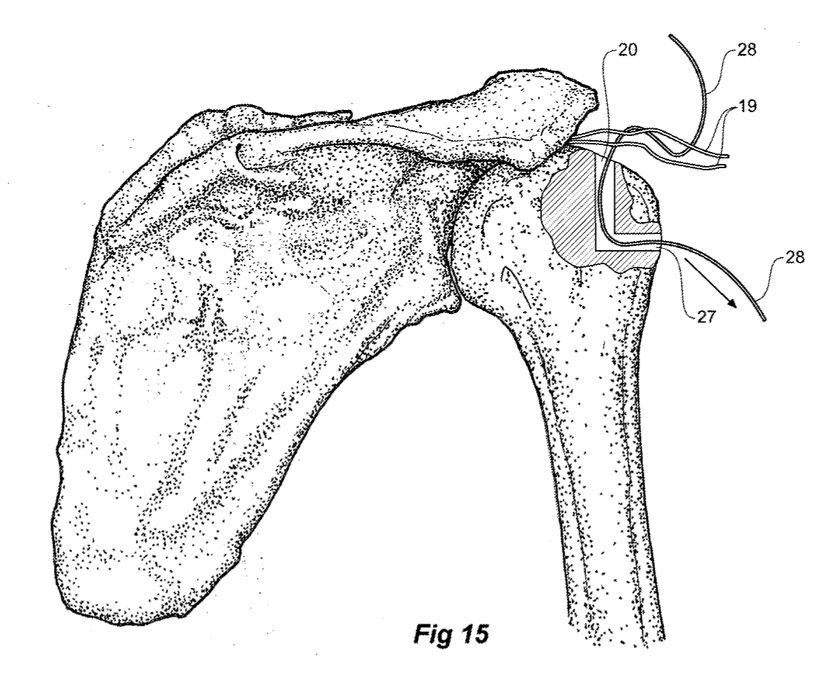

The suture 19 which is attached to a tendon of the rotator cuff muscle group is then attached to the suture or guide thread 28 at its upper end, by either an in-line knot, or by passing the suture 19 through a braided snare section in the suture or guide wire 28 (FIG. 15).

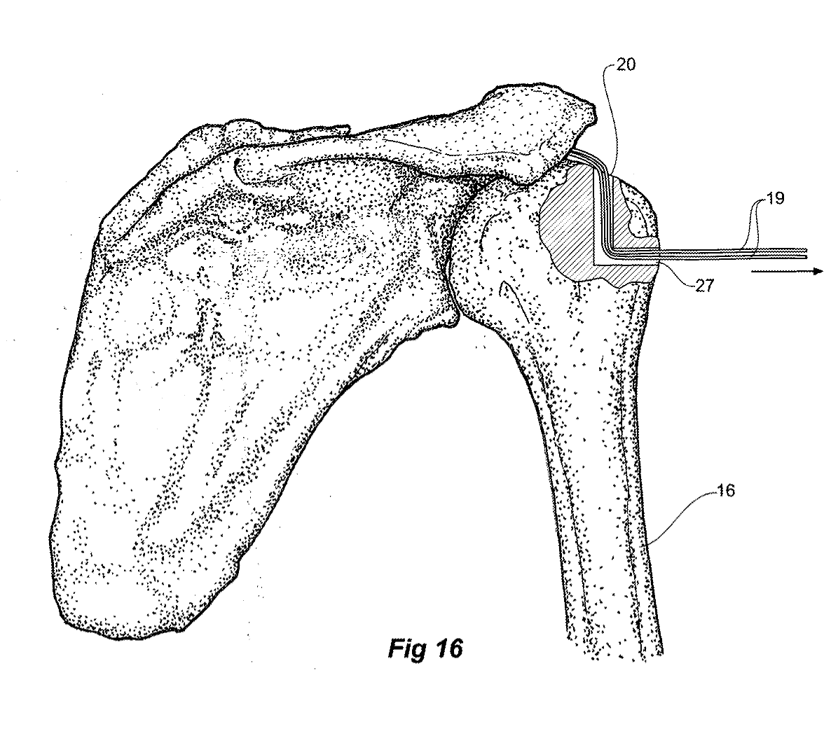

The suture or guide wire 28 is drawn in a distal direction by pulling on the suture or guide wire 28 from externally of the lower skin aperture 29. The suture 19 is attached at the other end of the suture to a tendon of the rotator cuff muscle group. The suture and attached tendon is thus drawn back through to the first skin aperture 18 and further through the created bone tunnel beginning at 20. The sutures path then follows the tunnel and exits the body via the lower skin aperture 29 and external to the patient as illustrated in FIG. 16 (which does not show the tendon attached to the other end of the suture).

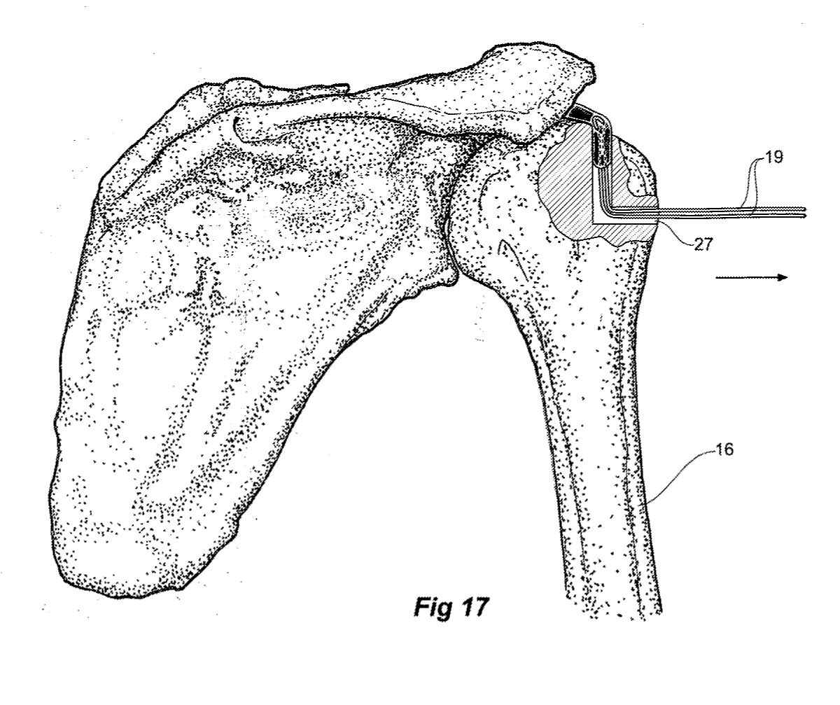

As illustrated in FIG. 17 by applying a further pulling force to the free end of the suture 19 from outside the body (which is an ideal location from which to do so, since there is space and leverage available, as opposed to doing so arthroscopically from within the body) the attached tendon is drawn a short distance into the first hole 20 in the numerous bone 16, as the main purpose of the force applied by the suture is to bring the tendon into contact with a prepared bone surface so they can create a lasting union by natural means.

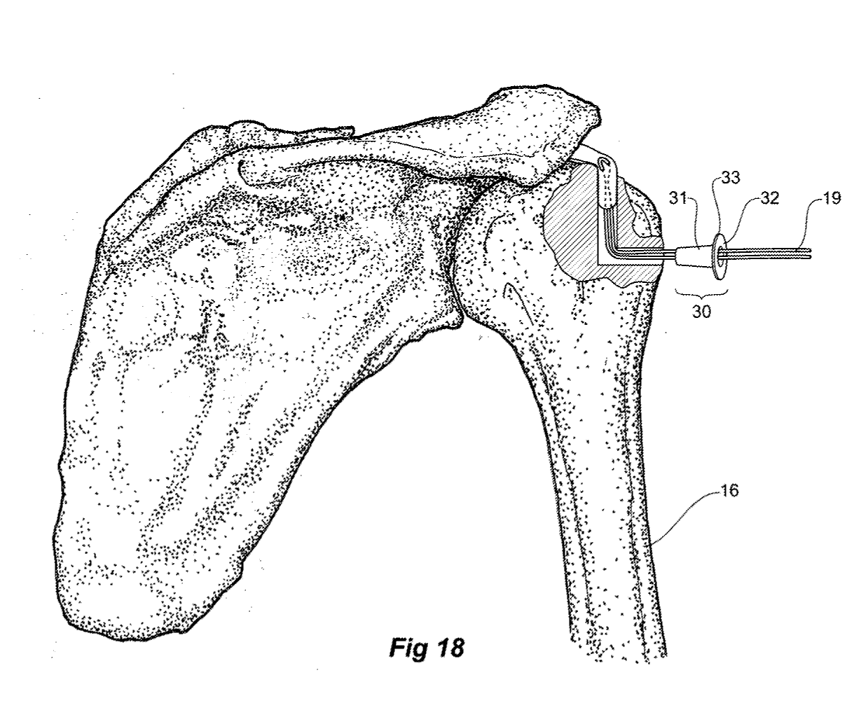

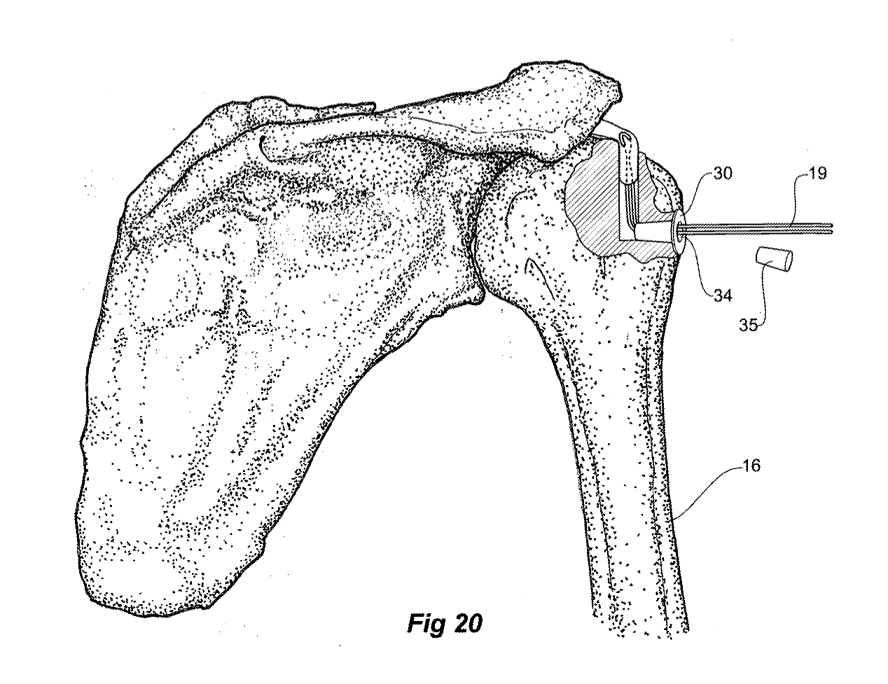

FIGS. 18 and 20 depict an arthroscopic transosseous knotless suture anchoring arrangement, including a hollow body and a suture holding element. The hollow body may also be referred to in this specification as an anchor.

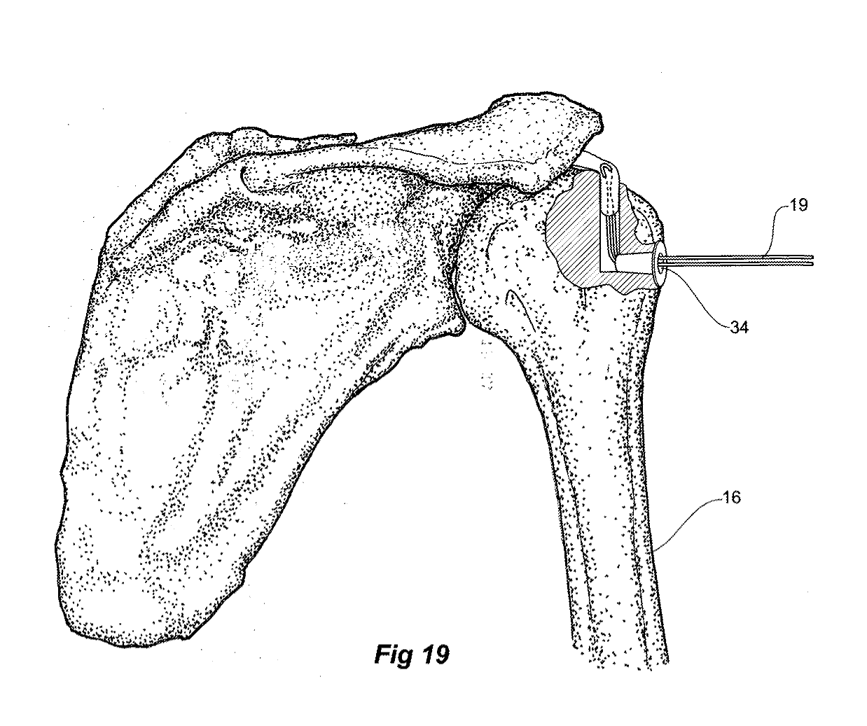

The hollow body 30 has an outer surface 31 which abuts the inside of the aperture 27 located in the cortical bone of the humerus 16 and thus is at least partially inserted into the cortical bone. In one embodiment one end of the hollow body 30 there is a rim 33 which radially extends from the hollow body. The rim 33 is adapted to receive a force on an outer (with respect to the bone and patient) by the provision in one embodiment of a force application surface 32 and thus the inner facing portion of the rim 33 (underside of the rim) to abut the outer surface of the humerus bone 16 adjacent and surrounding the bony cavity of second hole 27. FIGS. 19 and 20 only pictorially illustrate the hollow body 30 located in the humerus bone 16 with the free end 19 of the suture leading out of the centrally located aperture 34 (FIG. 19) of the hollow body 30 and the suture still visible within the bone, which if drawn true to life would not be the case.

FIG. 20 depicts a holding element 35 which is shaped and sized for fitment with and within the hollow of the body 30 which when fitted therein holds a portion of a suture 19 located within the hollow body with respect to the cortical bone within which the hollow body is at least partial inserted into.

The holding element 35 can also be shaped and sized for interference fitment with and within the hollow of the body 30 which may be achieved by slightly oversizing the holding element with respect to the interior shape of the hollow in the hollow body. The holding element 35 can also be shaped and sized for interference fitment with and within the hollow of the body 30 by having closely complementary shapes of the outside of the holding element and the inside of the hollow element still slightly oversizing the holding element with respect to the interior shape of the hollow in the hollow body but he surfaces are not planar and could include tangs and tabs with sufficient give to allow for one way fitment.

The holding element 35 can also be shaped and sized for removeable fitment with and within the hollow of the body 30 which may be achieved using the oversizing arrangement described above or by allowing one way fitment and a reverse release arrangement, such as tangs and tabs which interact to provide such engagement and release into and out of the interior shape of the hollow in the hollow body.

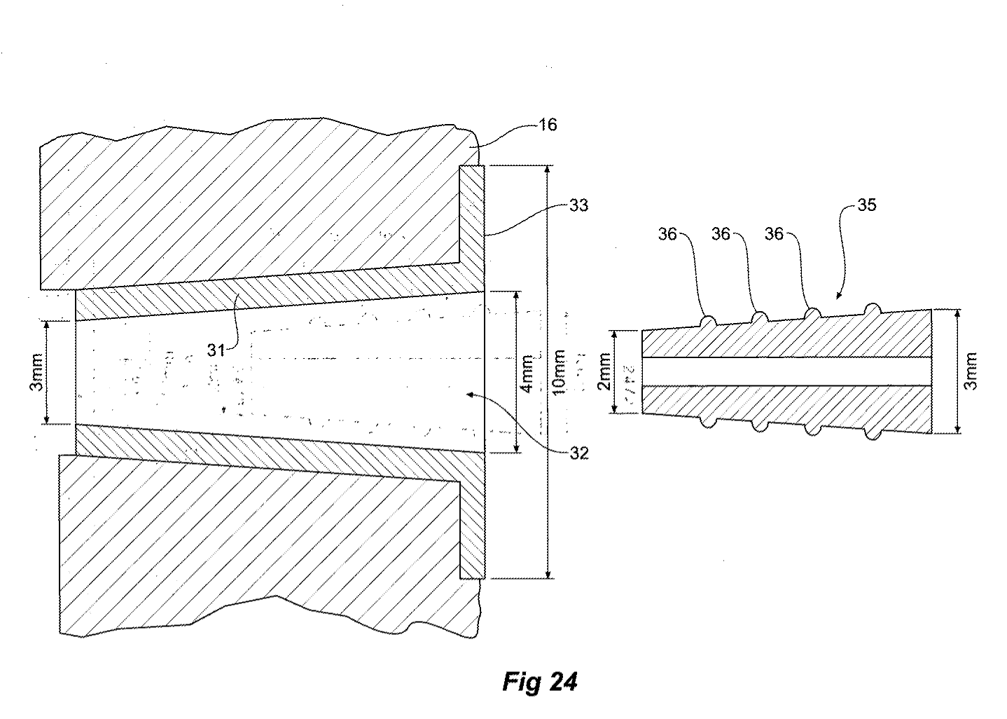

A shape provided in a preferred embodiment of the invention consists of the hollow in the body 30 in the form of a single wall of circular cross-section along the longitudinal axis of the body 30 having its largest internal diameter on the rim end of the body and its smallest internal diameter at the other end of the body. Thus, in a preferred embodiment the holding element 35 preferably has a complementary shape, so as to provide an interference fitment of the holding element to the hollow body sufficient to retain the holding element within the hollow body and also accommodate and secure at least a portion of a suture material within the hollow body (that is passing through the hollow body) at the same time. In another embodiment the circular cross-section of the internal hollow of the body and the outer surface of the holding element varies in diameter so as to taper (that is the radius varies at a constant rate along the length so that the side walls of both the hollow and the outer surface of the holding element are straight in lateral cross-section as illustrated at least in respect to the hollow body shown in FIG. 24. While the outer surface of the holding element 35 is shown with ridges 36 in that same figure, more detail of which will be provided later in the specification.

Furthermore, the holding element 35 should be removable from the hollow body 30 if required. There can be numerous ways by which the holding element can be removed, one of which includes the provision of an engagement surface on the surface which remains exposed to the surgeon even once the holding element has been fitted to hollow body. Furthermore, if no such engagement surface as explained above is provided to the holding element 35 then other extraction techniques which may be not unlike the extraction of a cork from a bottle, or others techniques can be employed as seen fit by the surgeon. In one example, the holding element 35 depleted in FIG. 24 is itself hollow and an appropriately shaped tool can be inserted into the hollow to assist removal of a hollow element from the hollow body.

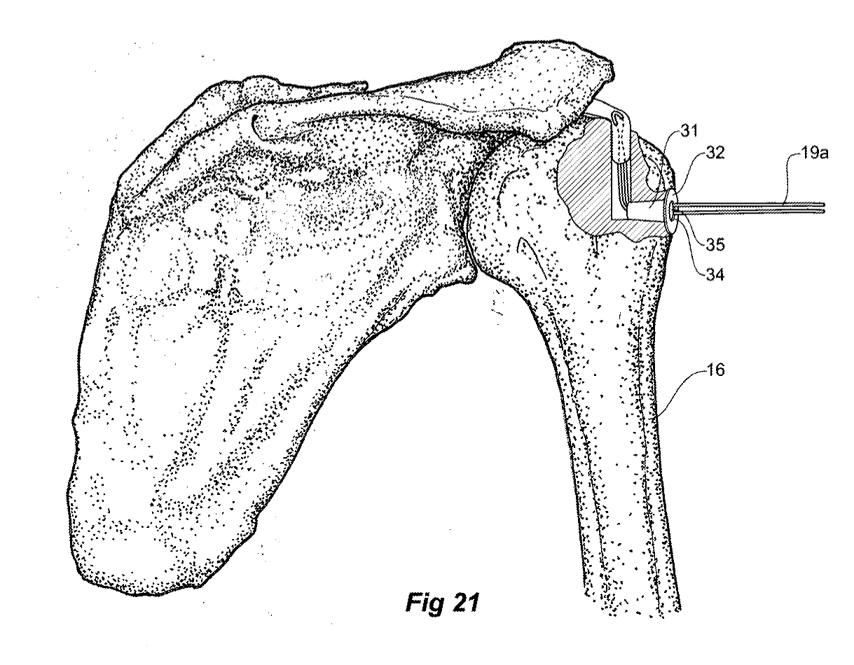

Pictorially illustrated in FIG. 21 is the result of the following steps. While a portion of the suture 19 is located within the hollow body 30 and while, the free end 19a of the suture 19 is under tension caused by a force on the suture applied, in a direction away from the outer surface of the cortical bone, that force being applied by the surgeon pulling on the free end of the suture 19 with a sufficient force to bring the tendon into adjacency with the prepared surface of the bone, another force in substantially the opposite direction is applied to the force receiving portion 32 of the rim 33 of the hollow body 30, the holding element 35 is interference fitted into the hollow body so as to capture a portion of the suture 19 under tension and while the inner facing portion of the rim 31 of the hollow body abuts the cortical bone portion. The captured portion of the suture is thus held in place with respect to the outer surface of the cortical bone, thus anchoring the sutured tendon to a stable portion of the body. The fixing of the hollow body to the cortical portion of the bone is also assisted by the tension between the captured suture and the rotor cuff tendency to retract towards the muscle.

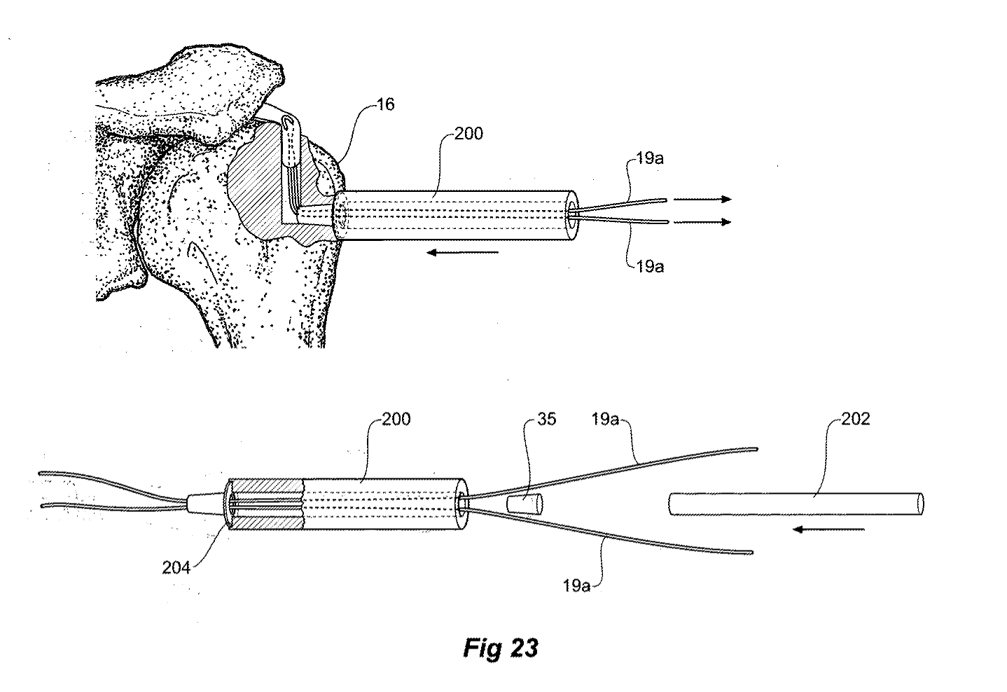

In an embodiment a tool or other suitable medical instrument can be used to apply the required, force to the holding element 33. FIG. 23 provides an illustration of one embodiment of such a tool. A holding element delivery device 200 includes an elongate body with an internal longitudinal aperture the length of the device having an internal diameter larger that the largest outer diameter of a holding element. In one embodiment there is provided a recessed shape 204 (one view of which is shown in FIG. 23) at one end of the holding element delivery device to allow interference fit of that end and temporary attachment to the periphery of the rim 32 of the hollow body 30, although this feature is only preferable for both holding the hollow body as it is inserted into the body and into the aperture in the bone and also to stabilise the union of the holding element delivery device and the partially inserted hollow body during the holding element fitment process.

After the suture 19 which is attached to a tendon of the rotator cuff muscle group is inserted, through the hollow body 30 and along the hollow body of the tool 200, the tool 200 is used to guide the hollow body 30 through the skin aperture 29 and then into the lateral surgical access aperture second hole 27 in the humerus bone 16, until the hollow body 30 contacts the outer cortical surface of the humerus with the underside of the rim 32.

The application of suitable pressure from external of the body to the tool 200 allows for the firm placement of the hollow body into the humerus bone 16 by the surgeon. With the guide in place, tension is placed on the suture 19 to ensure adequate advancement of the tendon of the rotator cuff muscle group into the first humeral hole 20. Once an adequate position of the rotator cuff tendon has been confirmed typically using arthroscopic inspection, the holding element 35 is inserted into the open end of the guide 200 and pushed along the length of the holding element delivery device 200 with a holding element placement device 202, while as mentioned previously maintaining appropriate tension on the free end of the suture 19.

The holding element placement device 202 in one embodiment has a length, and an outer diameter less than the smallest internal diameter of the holding element delivery device 200.

The holding element delivery device has an end shaped to conform to at least a portion, of the shape of the end of the hollow body external, of the cortical portion of a bone. In another embodiment of the invention the holding element placement device includes at least one longitudinal channel on its outer surface shaped and sized to at least accommodate a suture.

In yet a further embodiment the holding element placement device includes at least two longitudinal channels on its outer surface shaped and sized to at least accommodate a suture in each channel.

A desirable position of the two strands of the suture 19 is on opposite sides of the holding element 35, and a feature of the holding element placement device 202 is a channel on either side (not shown) to ensure the suture 19 stands are correctly positioned. The holding element placement device is provided with a mark or indicia 202a which indicate the length of the shaft that needs to be inserted into the holding element delivery device 200 so as to ensure that the holding element 35 is located within the hollow body 30. The amount offeree applied will be a judgement call by the surgeon.

FIG. 24 provides cross-sectional views of the hollow body 30 located in a cortical portion of a bone 16 and a holding element 35. The relative dimensions of the body and element are merely illustrative and should not limit the scope of the invention.

The materials of which the hollow body and holding element are made are preferably biocompatible and may not be of the same material, colour, density, or hardness.

The shape of the outer surface as shown in cross-section of an embodiment of the holding element 35 indicates the use of a plurality of ridges 36 along the length of the holding element. The ridges are only shown in cross-section and may extend all or only part the way about the outer surface of the element. At least a portion of one or more of the ridges contribute to the interference fit forces which restrain the dislodgement of the holding element from the hollow body as well as apply a holding force to the captured portion of the suture 19. Use of ridges of the configuration described above, or the use of ridges at all, is not essential as long as both forms of holding force are provided. It will be noted that the outline cross-sectional shape of the holding element substantially conforms to the inside shape of the hollow body which contributes to the application of the forces mentioned above. In this embodiment of the holding element 35 it is hollow but this also is not an essential feature. As disclosed previously in this specification the fitment may be of any type including of the interference type, the engagement type, and one or other of the fitments may allow the holding element to be removeable from the hollow body.

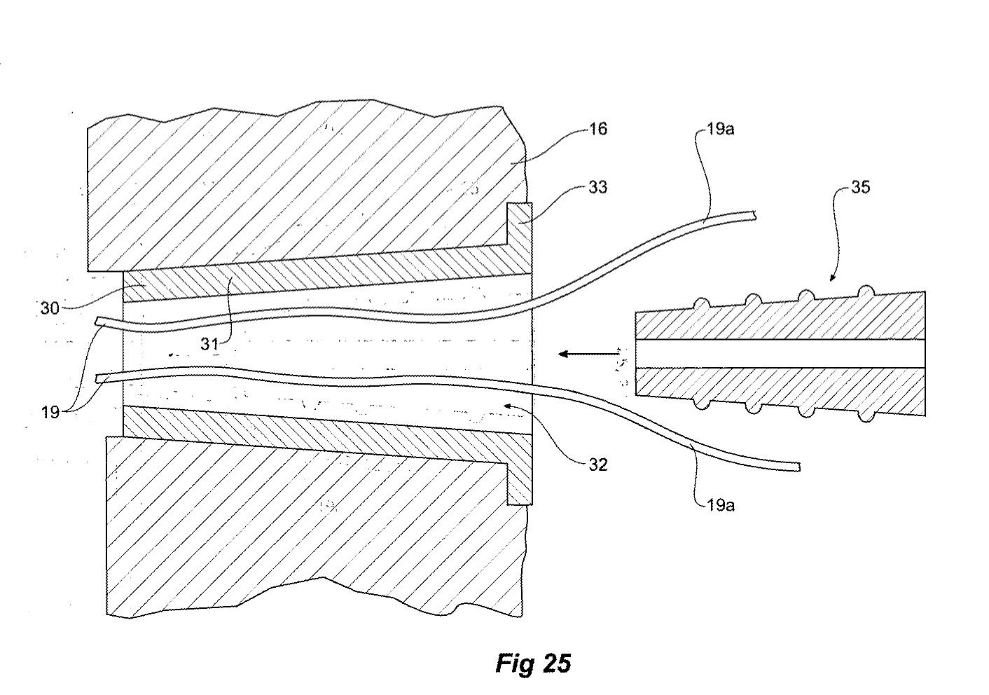

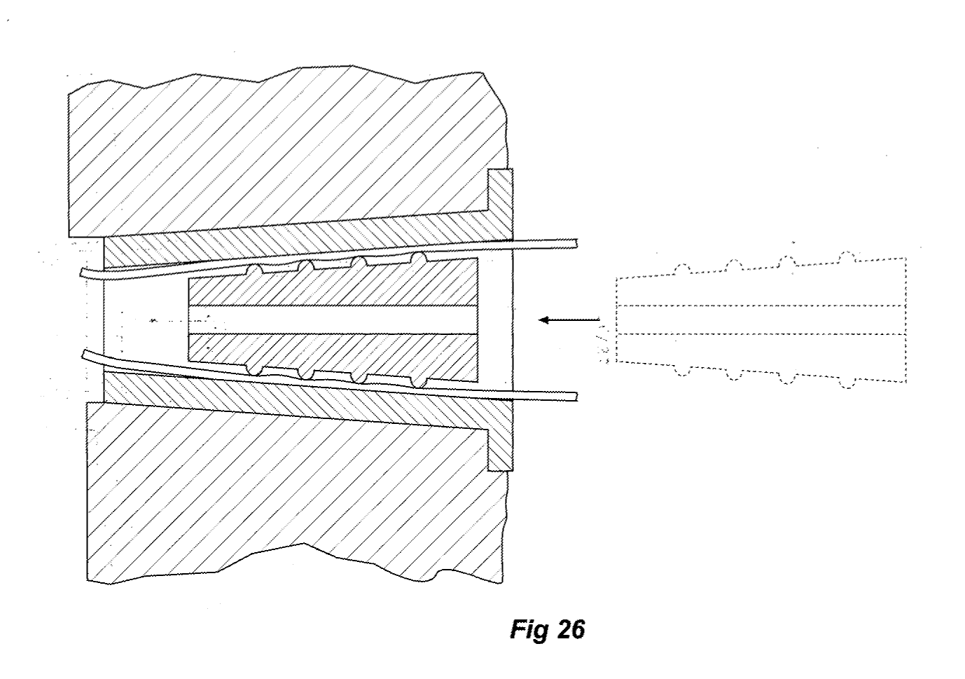

FIG. 25 depicts the bone 16 and the suture 39 and it's free end of 19a with the holding element 35 external of the hollow body 30. FIG. 26 is merely illustrative of the fitment of the holding element 35 into the hollow body 30.

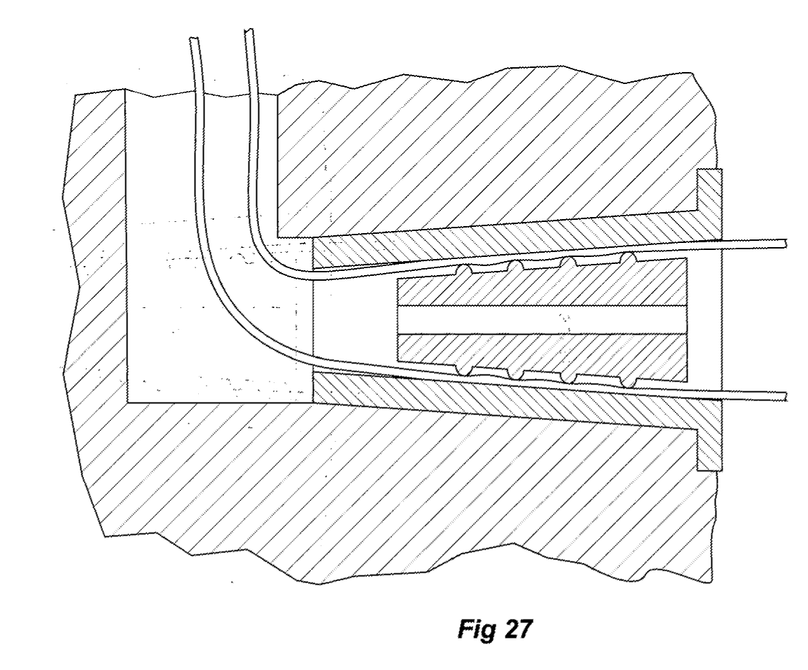

FIG. 27 depicts a placement configuration of the holding element 35 and hollow body 30 showing the capture of the suture 19 therein and the removal of the free ends of the suture 19a. Such an arrangement, as illustrated, provides a substantially flat surface on the outer surface of the bone which is an improvement if not an alternative for the prior termination techniques for sutures, which required the free ends of the sutures to he tied off and thereby secured with multiple knots, which are proud of any associated anchor.

FIG. 27 shows the surgical result, of the use of a device for capturing and holding a suture under tension with respect to a cortical bone.

Surgical recovery is prescribed for each surgical patient and generally includes some weeks of the arm being kept in a sling to immobilize the repaired region as much as possible. Further treatment some weeks later includes physical therapy so as to increase the range of motion of the shoulder while minimising load bearing activity so as to allow the bonding of the rotator cuff to the bone not to he unduly disturbed. Yet further treatment some further weeks later includes further physical therapy to increase the range of motion and initial strength training but full load bearing is still not possible. About six months later the repair should be completed sufficient for normal activities to commence.

It will be appreciated by those skilled in the art that the invention is not restricted in its use to the particular application described. Neither is the present invention restricted in its preferred embodiment with regard to the particular elements and/or features described or depicted herein. It will be appreciated that various modifications can be made without departing from the principles of the invention. Therefore, the invention should be understood to include all such modifications within its scope.

1. An arthroscopic transosseous knotless suture anchoring arrangement for holding a suture with respect to a cortical portion of a bone, comprising:
a hollow body for at least partial insertion into a cortical portion of a bone; and
a holding element having an elongate body sized for fitment with and within the hollow of the body tor holding a portion of a suture located in the hollow body with respect to the cortical portion of the bone.

2. An arthroscopic transosseous knotless suture anchoring arrangement according to claim 1, wherein the holding element and hollow body are adapted for interference fitment together.

3. An arthroscopic transosseous knotless suture anchoring arrangement according to claim 1, wherein the holding element and hollow body are adapted for removeable fitment together.

4. An arthroscopic transosseous knotless suture anchoring arrangement according to claim 1, wherein the hollow body further includes a rim radially extending from an end of the body.

5. An arthroscopic transosseous knotless suture anchoring arrangement according to claim 1, wherein the hollow varies in diameter being at least wider at one end to the other end of the body.

6. An arthroscopic transosseous knotless suture anchoring arrangement according to claim 1 wherein the hollow tapers in diameter being at least wider at one end to the other end of the body.

7. An arthroscopic transosseous knotless suture anchoring arrangement according to claim 1, wherein the hollow body has an internal surface shaped to assist the fitment of the holding element therein.

8. An arthroscopic transosseous knotless suture anchoring arrangement according to claim 1, wherein the holding element has an outer surface shaped to assist the fitment of the holding element in the hollow body.

9. An arthroscopic transosseous knotless suture anchoring arrangement according to claim 1, further including comprising:
a holding element delivery device having an elongate body with an internal longitudinal aperture the length of the device having an internal diameter larger than the largest outer diameter of the holding element and the holding element delivery device further adapted, to guide the hollow body into the body and at least partially into a cortical portion of a bone.

10. An arthroscopic transosseous knotless suture anchoring arrangement according to claim 9, further comprising:
a holding element placement device having a length, and an outer diameter less than the smallest internal diameter of the holding element delivery device,
wherein the holding element delivery device is adapted to guide a holding element into the hollow body when the holding element placement device is inserted into the holding element delivery device after the holding element.

11. An arthroscopic transosseous knotless suture anchoring arrangement according to claim 9, wherein the holding element delivery device has an end shaped to conform to at least a portion of the shape of the end of the hollow body external of the cortical portion of a bone.

12. An arthroscopic transosseous knotless suture anchoring arrangement according to claim 9, wherein the holding element placement device includes at least one longitudinal channel on an outer surface shaped and sized to at least accommodate a suture.

13. An arthroscopic transosseous knotless suture anchoring arrangement according to claim 12, wherein the holding element placement device includes at least two longitudinal channels on an outer surface shaped and sized to at least accommodate a suture in each channel.

14. An arthroscopic transosseous knotless suture anchoring arrangement according to claim 10, wherein the holding element placement device includes a mark or indicia which indicates the length of the device that needs to he inserted into the holding element delivery device so as to ensure that the holding element is in fitment with and within the hollow body.

15. A device for capturing and holding a suture under tension with respect to a cortical bone portion comprises:
a hollow elongate body having an inner surface forming the hollow, at least a portion of the body for placement in a cavity located in the cortical bone portion, the hollow body having a rim radially extending from, one end of the body, the rim adapted to receive a force that forces at least a portion of the rim to abut the cortical bone adjacent to the cavity in the cortical bone; and
a holding element sized for fitment with and within the hollow of the body;
wherein a portion of the suture is located within the hollow body and while, the free end of the suture is under tension caused by a force on the suture applied in a direction away from the outer surface of the cortical bone, and another force in substantially the opposite direction is applied to the rim of the body, the holding element is interference fitted into the hollow body so as to capture a portion of the suture under tension which is located within the hollow body while the rim of the body abuts the cortical bone portion and also holds the captured portion of the suture with respect to the cortical bone portion.
